SEQUENCE LISTING (31 sequences)

SEQ ID NO: 1 - protein, 36 aa, Homo sapiens
GSRLLHSDYMNMTPRRPGPTRKHYQPYAPPRDFAGS

SEQ ID NO: 2 - protein, 26 aa, Homo sapiens
GSMIETYNQTSPRSAATGLPISMKGS

SEQ ID NO: 3 - protein, 30 aa, Homo sapiens
GQNQLYNELNLGRREEYDVLDKRRGRDPEM

SEQ ID NO: 4 - protein, 30 aa, Homo sapiens
RKNPQEGLYNELQKDKMAEAYSEIGMKGER

SEQ ID NO: 5 - protein, 27 aa, Homo sapiens
RGKGHDGLYQGLSTATKDTYDALHMQA

SEQ ID NO: 6 - protein, 27 aa, Homo sapiens
YEKSDGVYTGLSTRNQETYETLKHEKP

SEQ ID NO: 7 - protein, 31 aa, Homo sapiens
GNKBPEDRVYEELNIYSATYSELEDPGEMSP

SEQ ID NO: 8 - protein, 31 aa, Homo sapiens
KQTLLPNDQLYQPLKDREDDQYSHLQGNQLR

SEQ ID NO: 9 - protein, 31 aa, Homo sapiens
ALLRNDQVYQPLRDRDDAQYSHLGGNWARNK

SEQ ID NO: 10 - protein, 33 aa, Homo sapiens
QNKERPPPVPNPDYEPIRKGQRDLYSGLNQRRI

SEQ ID NO: 11 - protein, 28 aa, Homo sapiens
HVDNEYSQPPRNSRLSAYPALEGVLHRS

SEQ ID NO: 12 - protein, 33 aa, Homo sapiens
PPRTCDDTVTYSALHKRQVGDYENVIPDFPEDE

SEQ ID NO: 13 - protein, 33 aa, Homo sapiens
EYEDENLYEGLNLDDCSMYEDISRGLQGTYQDV

SEQ ID NO: 14 - protein, 32 aa, Homo sapiens
KAGMEEDHTYEGLDIDQTATYEDIVTLRTGEV

SEQ ID NO: 15 - protein, 33 aa, Homo sapiens
PLPNPRTAASIYEELLKHDTNIYCRMDHKAEVA

SEQ ID NO: 16 - protein, 27 aa, Homo sapiens
YEKSDGVYTGLSTRNQETYDTLKHEKP

SEQ ID NO: 17 - protein, 26 aa, Homo sapiens
GQDGLYQELNTRSRDEYSVLEGRKAR

SEQ ID NO: 18 - protein, 27 aa, Homo sapiens
GQDGLYQELNTRSRDEAYSVLEGRKAR

SEQ ID NO: 19 - protein, 28 aa, Homo sapiens
GQDGLYQELNTRSRDEAAYSVLEGRKAR

SEQ ID NO: 20 - protein, 29 aa, Homo sapiens
RKNPQEGLYNELQKDKMAEDTYDALHMQA

SEQ ID NO: 21 - protein, 30 aa, Homo sapiens
GQNQLYNELQQQQQQQQQYDVLRRGRDPEM

SEQ ID NO: 22 - DNA, 129 bp, Homo sapiens
cgggaagcttccaccatgggggccgcacgcagcccgccgtccgctgtcccggggcccctgctggggctgctcctgctgctcctgggcgtgctggccccgggtggtgcctccctgcgactcctggaccac

SEQ ID NO: 23 - DNA, 36 bp, Homo sapiens
cccggcgccccacaggggcatgtagtccggaattgg

SEQ ID NO: 24 - protein, 323 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGAG

SEQ ID NO: 25 - protein, 292 aa, Homo sapiens
SLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWGAG

SEQ ID NO: 26 - protein, 789 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTV

SEQ ID NO: 27 - protein, 770 aa, Homo sapiens
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTV

SEQ ID NO: 28 - protein, 27 aa, Homo sapiens
FWVLVVVGGVLACYSLLVTVAFIIFWV

SEQ ID NO: 29 - protein, 41 aa, Homo sapiens
RSKRSRLLHSDYMNMTPRRPGPTRKHYQPYAPPRDFAAYRS

SEQ ID NO: 30 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 186 aa, Homo sapiens
FWVLVVVGGVLACYSLLVTVAFIIFWVTRGSRSKRSRLLHSDYMNMTPRRPGPTRKHYQPYAPPRDFAAYRSGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2009/0123944 A1
(43) Pub. Date: May 14, 2009

(54) Bioassays

(75) Inventors: Helene Margaret Finney, Berkshire (GB); Alastair David Griffiths Lawson, Berkshire (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Bruxelles (BE)

(21) Appl. No.: 12/094,660

(22) PCT Filed: Nov. 21, 2006
(86) PCT No.: PCT/GB2006/004332

§ 371 Date: Oct. 21, 2008

(30) Foreign Application Priority Data
Nov. 24, 2005 (GB) 0523954.6

(51) Int. Cl. G01N 33/53; C12N 15/00; C12N 5/06; C07K 2/00

(52) U.S. Cl. 435/7.21; 435/320.1; 435/325; 530/300

(57) ABSTRACT

The invention relates to vectors encoding bioassay receptors and in vitro bioassays using said bioassay receptors for assessing compounds of interest. In particular, the bioassays provide a generic platform for the comparison of binding of different ligands to their respective receptors or binding partners in the presence and/or absence of a compound of interest.

The invention disclosed herein relates to vectors encoding bioassay receptors and in vitro bioassays using said bioassay receptors for assessing compounds of interest. In particular, the bioassays provide a generic platform for the comparison of binding of different ligands to their respective receptors or binding partners in the presence and/or absence of a compound of interest.

Antibodies have long been viewed as potential agents for therapeutic interventions via targeted drug delivery, largely with a view to exploiting the combination of high specificity and affinity of the antibody-antigen interaction. Antibody binding may be measured by assaying the output of a bioassay on binding of the antibody to an antigen, e.g. a ligand or its receptor. Thus, conventional in vitro bioassays are dependent on, and limited by, the number of endogenous cell surface-expressed receptors. Indeed, an appropriate cell line expressing the selected receptor must be identified. Furthermore, receptor numbers vary hugely depending on the cell type and the receptor in question. Potential therapeutic antibodies must be assessed by a bioassay. However, typically, a different assay is developed for each product. Many assays have long read-outs giving a large potential for error. Appropriate bioassay read-outs vary widely in type, time point and the level of understanding of the biology involved. Comparison of such assays with different read-outs is thus, generally, meaningless. A generic assay platform would allow the development of a robust assay format that is essentially similar for each potential therapeutic antibody product, thereby facilitating a greater understanding of product stability and permitting direct, meaningful comparison of different batches of product. Such a generic assay platform is useful for the initial screening for a product, analysis of the potency of a product, monitoring of stability and batch reproducibility of a product. Most advantageously, such a generic assay platform would allow comparison of the efficacy of different antibodies raised against the same antigen. A generic assay would also facilitate report submission and regulatory authority understanding.

The invention disclosed herein overcomes the difficulties described above and provides a standardised in vitro bioassay using cells expressing recombinant receptors of interest. In particular, the assays of the invention provide a generic platform for assessing in vitro ligand binding using the bioassay receptors of the invention and permit the assessment of agents that either antagonise or agonise said binding.

Accordingly, provided is a vector encoding a bioassay receptor comprising:
(a) an extracellular ligand-binding region capable of binding to a ligand;
(b) a transmembrane region;
(c) one or more intracellular signalling regions capable of transmitting a signal wherein said extracellular ligand-binding region and intracellular signalling region are not naturally fused together; and
(d) a reporter region;
   wherein when said vector is expressed as a membrane-bound protein in a selected host cell under conditions suitable for expression, the binding of a ligand to the extracellular ligand-binding region results in the generation of a detectable signal from the reporter region.

The skilled artisan will understand that if a signal sequence is used to target the extracellular region to the cell surface of a host cell, said signal sequence will be present at the N-terminus of the bioassay receptor and followed by the extracellular ligand-binding region, transmembrane region and intracellular signalling region or regions. Optionally, one or more regions can be separated from an adjacent region by the incorporation of a spacer sequence in the DNA code. For example, during construction of the vector encoding the bioassay receptor, one or more restriction sites may leave one or more spacer amino acid residues between regions on splicing of the regions together. In one embodiment, each region is separated from its neighbouring regions by a spacer sequence. The spacer sequence may be 2 amino acids, or 3, 4, 5, 6, 7, 8, 9, 10, or more amino acids.

An extracellular ligand-binding region includes any protein (or nucleic acid encoding such a protein) that is capable of binding a ligand. Most preferably, the ligand is a soluble ligand. Membrane-bound extracellular ligand-binding regions of the bioassay receptors of the invention include surface membrane receptors, such as kinase receptors, G-protein coupled receptors (GPCRs), growth factor receptors, cytokine receptors such as interleukin receptors, e.g. IL-1R (Type I and II), IL-2R (α, β and γ subunits), IL-3R, IL-4R, IL-5R, IL-6R; gp130, IL-8R, IL-13Rα1, IL-4Rα, IL-15R (α, β and γ subunits), IL-17R; TNF receptor(s) (TNF-RI and TNF-RII); receptors for IL-β, IL-2, IL-10, IL-15, G-CSF, CSF-1, M-CSF, GM-CSF, HGF, EGF, PDGF, IGF, FGF, TGF-β, IP-10, ITAC, MIG and VEGF; CD markers such as CD2, CD4, CD5, CD7, CD8, CD11a, CD11b, CD11c, CD11d, CD16, CD19, CD20, CD22, CD24, CD28, CD33, CD40, CD48, CD69, CD70, CD122 and CD244; and receptors for ICOS-L, OX-40-L, CD40L and CD137-L. Membrane-bound extracellular ligand-binding regions of the bioassay receptors can also include molecules as binding regions that are not normally found on or within the cellular membrane, for example, a cytosolic or soluble protein, for example but without limitation, a protein which is normally secreted. Thus, extracellular ligand-binding regions can also include SOST, and LDL-related proteins such as LRP5 and LRP6, chemokines, cytokines, and growth factors which, by virtue of the transmembrane region, are presented extracellularly. It will be understood by those skilled in the art that a signal sequence targeting the extracellular ligand-binding region to the cell surface is included in the DNA sequence of the vector encoding the bioassay receptor. Such signal sequences are known in the art and include sequences that are naturally associated with the extracellular ligand-binding region (signal sequences/leader sequences). A signal sequence will generally be processed and removed during targeting of the extracellular ligand-binding region to the cell surface.

In one embodiment, an extracellular ligand-binding region can be chosen such that it interacts with one or more other extracellular ligand-binding regions to achieve multiply-associated extracellular ligand-binding regions capable of recognising (binding to) a ligand. Thus, in one embodiment, the bioassay receptor of the invention comprises more than one membrane-bound extracellular ligand-binding region. More preferably, two or more bioassay receptors comprising different ligand-binding regions may be expressed in a host cell to achieve multiply-associated extracellular ligand-binding regions capable of recognising (binding to) a ligand.

A transmembrane region generally serves to anchor the bioassay receptor to the cell membrane (thus the extracellular ligand-binding region is membrane-bound) and includes any protein (or nucleic acid encoding such a protein). Such a region can be derived from a wide variety of sources such as all or part of the alpha, beta, or zeta chain of the T cell receptor (TCR), CD28, CD4, CD5, CD8, CD3ε, CD16, CD22, CD23, CD45, CD80, CD86, CD64, CD9, CD37, CD122, CD137 or CD154, a cytokine receptor such as an interleukin receptor, TNF-R, a tyrosine kinase receptor or interferon receptor, or a colony stimulating factor receptor. Alternatively, the transmembrane region may be synthetic. Suitable synthetic transmembrane regions will comprise predominantly hydrophobic amino acids such as leucine and valine.

An intracellular signalling region includes any protein (or nucleic acid encoding such a protein) that can participate in the generation of a signal that results in direct or indirect production of an intracellular messenger system. Particular intracellular messenger systems include one or more kinase pathways such as a tyrosine kinase pathway, a MAP kinase pathway, or protein kinase C pathway; a G-protein or phospholipase-mediated pathway; a calcium-mediated pathway; a cAMP- or cGMP-mediated pathway; or one or more pathways involving synthesis of one or more cytokines such as an interleukin, e.g. IL-2, or transcription factors such as NFκB, NFAT or AP-1. The intracellular signalling regions are most preferably selected such that they act cooperatively.

Intracellular signalling regions may be derived from one or more naturally-occurring protein signalling sequences. Suitable examples include without limitation sequences derived from the TCR such as part of the zeta, eta or epsilon chain and include the first (TCRζ1), second (TCRζ2) and third (TCRζ3) immunoreceptor tyrosine-based activation motifs (ITAMs) of the TCR zeta chain, FcRγ such as FcRIIIγ or FcRIγ, FcRβ such as FcRIβ; CD3γ; CD3δ; CD3ε; and CD5, CD22, CD79a, CD79b, or CD66d. Particularly preferred ITAMs include those derived from TCRζ1, TCRζ2, TCRζ3 and FcεRIγ; CD4; CD8; and the gamma chain of a Fc receptor. Further included are SB28 (GSRLLHSDYMNMTPRRPGPTRKHYQPYAPPRDFAGS; SEQ ID NO:1) and SB29 (GSMIETYNQTSPRSAATGLPISMKGS; SEQ ID NO:2); one or both of the GS linkers at either end of each sequence may be omitted. Intracellular signalling regions are particularly intended to include synthetic signalling regions such as a synthetic region based on sequences of any of the above intracellular signalling regions. Synthetic ITAMs and synthetic signalling regions and methods for making these include those described in WO 00/63372 and WO 00/132867 which are incorporated herein by reference in their entirety (see Table 1 for examples). Combinations of these signalling motifs may be used, as described in WO 00/63372 and WO 00/132867.

Also included are signalling components such as from a cytokine receptor such as IL-1β, TNF-RI and TNFRII, and interferon receptors; Toll-like receptors (TLRs) such as TLR4; a colony stimulating factor such as GMCSF; a tyrosine kinase such as ZAP-70, fyn, lyk, ltk, syk and binding components thereof such as Vav, Crk, LAT and Grb-2; phospholipase C and phosphoinositide-3 kinase; an adhesion molecule, e.g. LFA-1 and LFA-2, B29, MB-1, CD3 delta, CD3 gamma, CD5, CD2 and CD154, and co-stimulatory molecules such as CD28, ICOS, CD134, and CD137. Also included are immunoreceptor tyrosine-based inhibitory motifs (ITIMs). Particular examples of ITIMs include FcyR (such as FcyRIIB), CD22, EPOR, IL-2ssR and IL-3pR.

TABLE 1
Source and amino acid sequences of primary
signalling motifs of particular use in the invention.
The position of the consensus amino acid sequence
is emphasised in bold.

| | Intracellular | |
|---|---|---|
| Source | Signalling Region | Amino Acid Sequence |
| TCRζ1 | SB1^{a} | GQNQLYNELNLGRREEYDVLDKRRGRDPEM |
| | | (SEQ ID NO:3) |
| TCRζ2 | SB2^{a} | RKNPQEGLYNELQKDKMAEAYSEIGMKGER |
| | | (SEQ ID NO:4) |
| TCRζ3 | SB3^{a} | RGKGHDGLYQGLSTATKDTYDALHMQA |
| | | (SEQ ID NO:5) |
| FcRγ | SB4^{a} | YEKSDGVYTGLSTRNQETYETLKHEKP |
| | | (SEQ ID NO:6) |
| FcRβ | SB5^{a} | GNKBPEDRVYEELNIYSATYSELEDPGEMSP |
| | | (SEQ ID NO:7) |
| CD3γ | SB6^{a} | KQTLLPNDQLYQPLKDREDDQYSHLQGNQLR |
| | | (SEQ ID NO:8) |
| CD3δ | SB7^{a} | ALLRNDQVYQPLRDRDDAQYSHLGGNWARNK |
| | | (SEQ ID NO:9) |
| CD3ε | SB8^{a} | QNKERPPPVPNPDYEPIRKGQRDLYSGLNQRRI |
| | | (SEQ ID NO:10) |
| CD5 | SB9^{a} | HVDNEYSQPPRNSRLSAYPALEGVLHRS |
| | | (SEQ ID NO:11) |
| CD22 | SB10^{a} | PPRTCDDTVTYSALHKRQVGDYENVIPDFPEDE |
| | | (SEQ ID NO:12) |
| CD79a | SB11^{a} | EYEDENLYEGLNLDDCSMYEDISRGLQGTYQDV |
| | | (SEQ ID NO:13) |
| CD79b | SB12^{a} | KAGMEEDHTYEGLDIDQTATYEDIVTLRTGEV |
| | | (SEQ ID NO:14) |
| CD66d | SB13^{a} | PLPNPRTAASIYEELLKHDTNIYCRMDHKAEVA |
| | | (SEQ ID NO:15) |
| FcRγ | SB4*^{a} | YEKSDGVYTGLSTRNQETYDTLKHEKP |
| | | (SEQ ID NO:16) |
| Non-natural | SB14^{a} | GQDGLYQELNTRSRDEYSVLEGRKAR |
| | | (SEQ ID NO:17) |
| Non-natural | SB15^{a} | GQDGLYQELNTRSRDEAYSVLEGRKAR |
| | | (SEQ ID NO:18) |
| Non-natural | SB16^{a} | GQDGLYQELNTRSRDEAAYSVLEGRKAR |
| | | (SEQ ID NO:19) |
| Non-natural | SBX^{a} | RKNPQEGLYNELQKDKMAEDTYDALHMQA |
| | | (SEQ ID NO:20) |
| Non-natural | SBQ9^{a} | GQNQLYNELQQQQQQQQQYDVLRRGRDPEM |
| | | (SEQ ID NO:21) |

As the skilled artisan will appreciate, amino acid deletions, insertions and/or mutations may be made from natural sequences in order to modify the precise nature of the regions, in accordance with what is required from the bioassay receptor.

As will be clear to the skilled artisan, combinations of intracellular signalling regions can be used within separate bioassay receptors or can be in series within a single bioassay receptor. Most preferably, they are in series.

In one embodiment, the intracellular signalling regions of the bioassay receptor comprise one intracellular signalling region derived from CD28 and a second intracellular signalling region derived from a component of the TCR, e.g. TCRζ, or derived from a component of the signalling cascade associated with said signalling region. In another embodiment, one intracellular signalling region is derived from CD28 and a second intracellular signalling region results in the direct or indirect generation of a messenger.

Most preferred combinations of intracellular signalling regions include CD28 and TCRζ, ICOS and TCRζ, CD134 and TCRζ, and CD28 and a synthetic signalling region derived from one or more ITAMs as described above.

A reporter region includes compounds capable of generating a measurable (detectable) signal, such as and without limitation, luciferase, secreted alkaline phosphatase (SEAP), green fluorescent protein or red fluorescent protein. Thus, reporter region signal measurements include measurement of light emission, fluorescence or alkaline phosphatase production. Most preferably, the signal measured is luciferase production or SEAP production. The signal used for assessing the compound of interest may also be measured using means known in the art depending on the signal generated by an intracellular signalling region. Preferred signals include measurement of cytokine production (e.g. IL-2 production), cell proliferation or apoptosis.

In one preferred embodiment, a vector for expressing a bioassay receptor of the invention comprises a DNA sequence encoding the extracellular region of IL-17R, a CD28 transmembrane region, a CD28 intracellular signalling region and TCRζ signalling region, and a reporter region encoding luciferase. In another preferred embodiment, the vector encoding a bioassay receptor comprises a DNA sequence encoding the extracellular binding region of KDR (VEGF receptor), a CD28 transmembrane region, a CD28 intracellular signalling region and TCRζ signalling region, and optionally a reporter region encoding luciferase.

In a most preferred embodiment, the vector of the methods of the invention comprises DNA encoding a transmembrane region derived from CD28 (SEQ ID NO:28), intracellular signalling regions derived from CD28 (SEQ ID NO:29) and the zeta chain of the TCR (SEQ ID NO:30), and an extracellular ligand-binding region derived from TNF-α, IL-17 receptor or KDR. The CD28 transmembrane and intracellular signalling regions and the zeta chain of the TCR may be linked with spacer sequences as shown in SEQ ID NO:31. Preferably, the reporter region is luciferase or SEAP. Most preferably, the DNA encodes an extracellular ligand-binding region amino acid sequence comprising or consisting of IL-17 receptor sequence of SEQ ID NO:24 or 25, or comprising or consisting of the KDR sequence of SEQ ID NO:26 or 27.

The invention further provides mammalian host cells comprising at least one vector encoding a bioassay receptor of the invention (i.e. a first vector). Host cells can be any cell or cell line which can be transfected or transformed and include Jurkat cells, HEK293, CHO, NIH3T3, NS0, Cos-7, Hela, MCF-7, HL-60, EL4, A549, and K562 cells. Most preferably, Jurkat cells are used.

In a preferred embodiment, the mammalian host cell comprises additional vector (i.e. different vector from the first vector of the invention) encoding a bioassay receptor comprising:
(e) an extracellular ligand-binding region;
(f) a transmembrane region;
(g) one or more intracellular signalling regions capable of transmitting a signal wherein said extracellular ligand-binding region and intracellular signalling region are not naturally fused together; and optionally
(h) a reporter region;
   wherein when said DNA sequence is expressed in a selected host cell under conditions suitable for vector expression, the binding of a ligand to the extracellular ligand-binding region results in the generation of a signal from the intracellular signalling region and, where present, a detectable signal from the reporter region. In one embodiment, the reporter region of part (h) is the same as that present in the first vector. Alternatively, the reporter region is different. Thus, for example, one reporter region can be luciferase and the other can be SEAP.

Most preferably, where a host cell comprises two bioassay receptors of the invention both comprising reporter regions, said regions are not the same. For example, one may be a luciferase reporter region and a second may be a SEAP reporter region. Alternatively, they may be the same reporter regions.

Mammalian host cells may be transfected with a vector of the invention using any convenient technique, such as electroporation, calcium phosphate transfection, DEAE-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al, Molecular Cloning: A Laboratory Manual, 2^{nd} Ed., Cold Spring Harbour laboratory Press, Cold Spring Harbour, N.Y., 1989). Suitable conditions for inducing vector expression within a host cell are well-known in the art. Transfection may be transient or, alternatively, cells lines stably expressing a bioassay receptor may be produced as known n the art. In particular, see Methods in Molecular Biology 7. Gene Transfer and Expression Protocols. Edited E. J. Murray 1991.

Accordingly, the invention further provides a polypeptide comprising:
(i) an extracellular ligand-binding region capable of binding to a ligand;
(j) a transmembrane region;
(k) one or more intracellular signalling regions capable of transmitting a signal wherein said extracellular ligand-binding region and intracellular signalling region are not naturally fused together; and
(l) a reporter region;
   wherein the binding of a ligand to the extracellular ligand-binding region results in the generation of a detectable signal from the reporter region. In one embodiment, the extracellular ligand-binding region is derived from a cytokine receptor, a cell surface receptor, or a soluble protein. In a preferred embodiment, the cytokine receptor is IL-17R. In another preferred embodiment, the cell surface receptor is KDR.

The invention also provides a mammalian cell comprising at least one vector encoding a bioassay receptor as defined in (a) to (d), above. Further provided is a mammalian cell comprising as a surface membrane protein, a polypeptide as described in (i) to (l), above. Preferably, the mammalian cell is a human cell, most preferably a Jurkat cell.

The invention also provides methods for assessing compounds of interest comprising use of any one or more of the vectors of the invention. Accordingly, provided is a method for assessing a compound of interest comprising:
(i) providing a mammalian cell comprising a vector of the invention encoding a bioassay receptor as defined in (a) to (d), above, and optionally, (e) to (h), above;
(ii) providing a first sample comprising a ligand;
(iii) providing a second sample comprising a compound of interest; and
(iv) measuring a signal generated by the intracellular signalling region or regions and/or a detectable signal generated by the reporter region or regions.

This method is particularly advantageous because it permits the development of a robust assay format that is essentially similar for each potential therapeutic antibody product, thereby facilitating a greater understanding of product stability and permitting direct, meaningful comparison of different batches of product. Previously, recombinant receptors have been disclosed as a means to facilitate a cell activation process wherein the activated cell could be used as a therapeutic by administration to a patient in need; see for example, WO 97/23613, WO 99/57268, EP0517895, WO 96/23814 and WO0132709; but there is no suggestion that the receptors described therein could be used in a robust and reproducible bioassay method.

In a most preferred embodiment, the vector of the methods of the invention comprises DNA encoding the amino acid sequence of a transmembrane region derived from CD28 (SEQ ID NO:28), intracellular signalling regions derived from CD28 (SEQ ID NO:29) and the zeta chain of the TCR (SEQ ID NO:30), and an extracellular ligand-binding region derived from TNF-α, IL-17 receptor or KDR. The CD28 transmembrane and intracellular signalling regions and the zeta chain of the TCR may be linked with spacer sequences as shown in SEQ ID NO:31. Preferably, the reporter region is luciferase or SEAP. Most preferably, the DNA encodes an extracellular ligand-binding region amino acid sequence comprising or consisting of IL-17 receptor sequence of SEQ ID NO:24 or 25, or comprising or consisting of the KDR sequence of SEQ ID NO:26 or 27.

In one embodiment, the methods of the invention additionally comprise the step of measuring a signal generated by the intracellular signalling region or regions and/or a detectable signal generated by the reporter region or regions before the provision of the second sample of part (iii), above.

A ligand includes any nucleic acid, protein or antigen of interest, for example but without limitation, CD marker ligands such as CD48, CD40L, CD40, CD122, cytokines and chemokines such as IL-2, IL-12, IL-13, IL-15, IL-17, IL-6, TNF-α, IL-β, IL-10, IP-10, ITAC, MIG, VEGF, co-stimulatory ligands such as ICOS-L, OX-40-L, CD137-L, components of signalling pathways, and indeed, any antigen of interest. A ligand also includes an antibody. In the methods of the invention, the ligand is most preferably a soluble ligand. The methods may also be used for a ligand presented on the surface of a mammalian cell, a bacterium, a virus, a yeast cell or other particle such as a synthetic particle, e.g. an agarose bead or a magnetic bead. It will be clear to one skilled in the art that such a ligand will have at least one binding partner that, for the purposes of the invention, is the extracellular ligand-binding region of the bioassay receptor that is expressed on the host cell surface.

The second sample comprises or consists of a compound of interest that competes with the ligand for binding to the extracellular ligand-binding region or, alternatively, binds to the ligand. Thus, a “compound of interest” includes antibodies, small molecules (e.g. NCEs) and other drugs, proteins, polypeptides and peptides, peptidomimetics, lipids, carbohydrates and nucleic acids. Most preferably, the compound of interest present within the second sample is an antibody. Thus, in one preferred embodiment, the second sample comprises antibodies that specifically interact with the extracellular ligand-binding region of the bioassay receptor and in a second preferred embodiment, the second sample comprises antibodies that specifically bind to the ligand. Specifically interacting with (e.g. recognising or binding to) means that the compound of interest, most preferably an antibody, has a greater affinity for the selected extracellular ligand-binding region, or ligand, than for other regions or ligands. The antibody may interact directly or indirectly with the extracellular ligand-binding region of the bioassay receptor. It will be understood that a second sample may also be used as a control, a standard, or a comparative sample. Thus, the methods of the invention may be performed more than once permitting comparison of samples.

The term ‘antibody’ as used herein includes complete antibodies and functionally active fragments or derivatives thereof and may be, but are not limited to, single chain antibodies, bi, tri or tetra-valent antibodies, Bis-scFv, diabodies, triabodies, tetrabodies, single domain antibodies, modified Fab fragments, Fab fragments, Fab′ and F(ab′)₂ fragments and epitope-binding fragments of any of the above (see for example Holliger and Hudson, 2005, Nature Biotech. 23(9): 1126-1136). In one example the antibodies are Fab′ fragments which possess a native or a modified hinge region. A number of modified hinge regions have already been described, for example, in U.S. Pat. No. 5,677,425, WO9915549, and WO9825971 and these are incorporated herein by reference. In another example the antibodies include those described in WO2005003169, WO2005003170 and WO2005003171.

Antibodies include immunoglobulin molecules and immunologically active portions of immunoglobulin molecules, i.e. molecules that contain an antigen binding site that specifically binds an antigen. The immunoglobulin molecules of the invention can be of any class (e.g. IgG, IgE, IgM, IgD or IgA) or subclass of immunoglobulin molecule. The constant region domains of the antibody, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required [e.g. antibody dependent cell-mediated toxicity (ADCC) and/or complement dependent cytotoxicity (CDCC). Alternatively, IgG2 and IgG4 isotypes may be used when the antibody molecule is intended for therapeutic purposes and antibody effector functions are not required.

The antibodies for use in the invention may be produced by any suitable method known in the art. Such antibodies include, but are not limited to, polyclonal, monoclonal, humanized, phage display derived antibodies or chimeric antibodies.

Monoclonal antibodies may be prepared by any method known in the art such as the hybridoma technique (Kohler & Milstein, Nature, 1975, 256:495-497), the trioma technique, the human B-cell hybridoma technique (Kozbor et al., Immunology Today, 1983, 4, 72) and the EBV-hybridoma technique (Cole et al., “Monoclonal Antibodies and Cancer Therapy”, pp. 77-96, Alan R. Liss, Inc., 1985).

Antibodies for use in the invention may also be generated using single lymphocyte antibody methods by cloning and expressing immunoglobulin variable region cDNAs generated from single lymphocytes selected for the production of specific antibodies by, for example, the methods described by Babcook, J. et al., 1996, Proc. Natl. Acad. Sci. USA, 93(15), 7843-7848, WO 92/02551, WO2004/051268 and WO2004/106377.

Chimeric antibodies are those antibodies encoded by immunoglobulin genes that have been genetically engineered so that the light and heavy chain genes are composed of immunoglobulin gene segments belonging to different species.

Humanized antibodies are antibody molecules having one or more complementarity determining regions (CDRs) from a non-human species and a framework region from a human immunoglobulin molecule (see, for example, U.S. Pat. No. 5,585,089).

The methods for creating and manufacturing recombinant antibodies are well known in the art (see for example, Boss et al., U.S. Pat. No. 4,816,397; Cabilly et al., U.S. Pat. No. 6,331,415; Simmons et al, 2002, Journal of Immunological Methods, 263, 133-147; Shrader et al., WO 92/02551; Orlandi et al., 1989, Proc. Natl. Acad. Sci. USA, 86, 3833; Riechmann et al., 1988, Nature, 322, 323; Queen et al., U.S. Pat. No. 5,585,089; Adair, WO91/09967; Mountain and Adair, 1992, Biotechnol. Genet. Eng. Rev, 10, 1-142; Verma et al., 1998, J. Immunol. Methods, 216:165-181; Holliger and Hudson, 2005, Nature Biotech. 23(9):1126-1136).

The antibodies for use in the present invention can also be generated using various phage display methods known in the art and include those disclosed by Brinkman et al., 1995, J. Immunol. Methods, 182:41-50; Ames et al., 1995, J. Immunol. Methods, 184, 177-186; Kettleborough et al. 1994, Eur. J. Immunol., 24, 952-958; Persic et al., 1997, Gene, 187, 9-18; and Burton et al., 1994, Advances in Immunol., 57, 191-280; WO 90/02809; WO 91/10737; WO 92/01047; WO 92/18619; WO 93/11236; WO 95/15982; and WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108.

Also, transgenic mice, or other organisms, including other mammals, may be used to produce antibodies (see for example U.S. Pat. No. 6,300,129).

The vectors of the invention will include all the necessary motifs and signals necessary for expression of the bioassay receptors of the invention in a host cell. Thus, a vector will comprise transcriptional initiation regions, a promoter region and a termination region. Promoter regions will be operably linked to the coding region comprising the bioassay receptor and may be inducible or constitutively active. Non-coding regions may be provided as desired, for example to stabilise mRNA. Nucleic acids encoding components of the bioassay receptors of the invention may be obtained using standard cloning and screening techniques, from a cDNA library derived from mRNA in human cells, using expressed sequence tag (EST) analysis (Adams, M. et al., 1991, Science, 252:1651-1656; Adams, M. et al., 1992, Nature 355:632-634; Adams, M. et al., 1995, Nature, 377:Suppl: 3-174). Nucleic acids encoding components of the bioassay receptors of the invention can also be obtained from natural sources such as genomic DNA libraries or can be synthesized using well known and commercially available techniques.

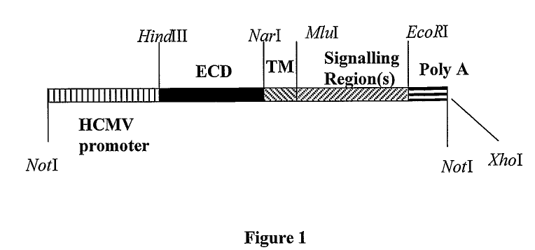

FIG. 1 shows the expression cassette cloned into the large NotI and XhoI restricted fragment of pBluescript® II SK(+) to generate the bioassay receptor shuttle vector.

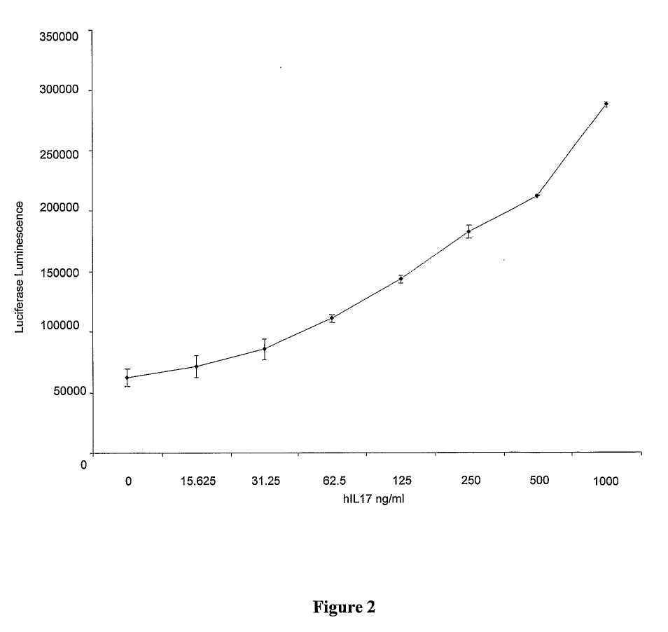

FIG. 2 shows the IL-17 induced luciferase response from IL-17R/CD28-TCRζ bioassay receptor.

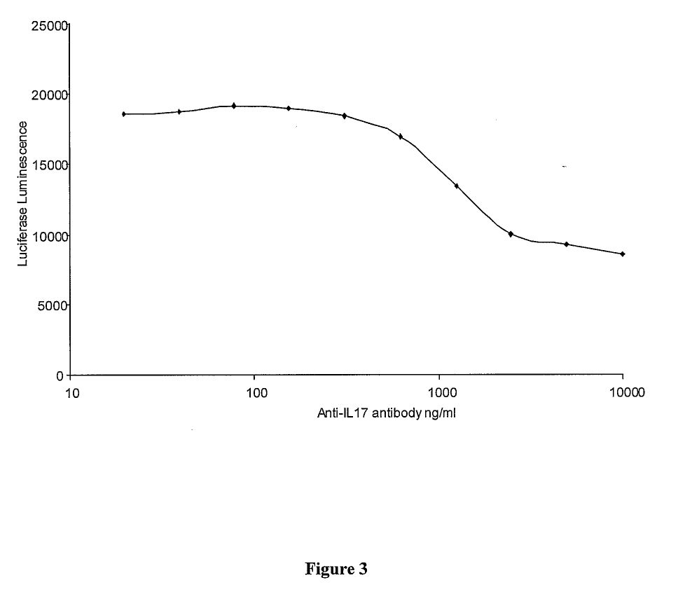

FIG. 3 shows anti-IL-17 antibody blocking of luciferase production in cells treated with a 500 ng/ml concentration of IL-17.

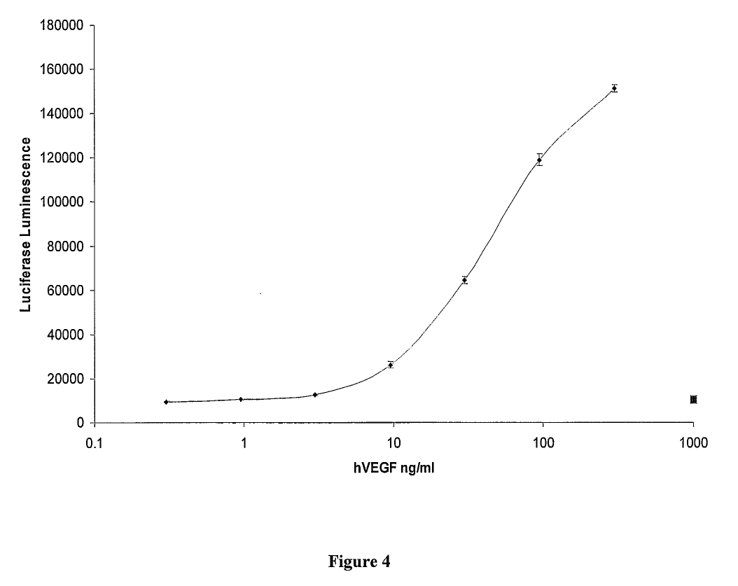

FIG. 4 shows the VEGF-induced luciferase response from KDR/CD28-TCRζ bioassay receptor. (▪) denotes cells treated in the absence of hVEGF.

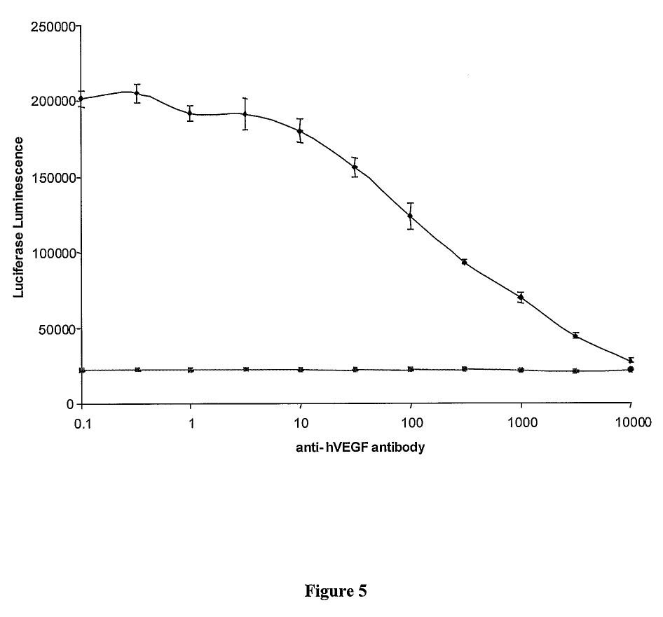

FIG. 5 shows anti-KDR antibody blocking of luciferase production in cells treated with a 200 ng/ml concentration of hVEGF (♦) and without hVEGF (▪). Cells treated in the absence of antibody, i.e. with hVEGF alone, and with medium alone are also shown (▴) and (), respectively.

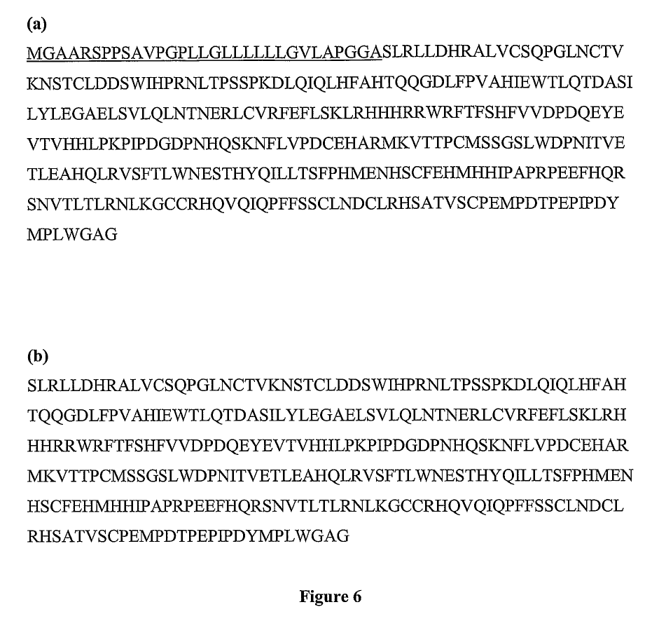

FIG. 6 shows the amino acid sequence of the IL-17 extracellular region with, panel (a) (SEQ ID NO:24), and without a leader sequence, panel (b) (SEQ ID NO:25).

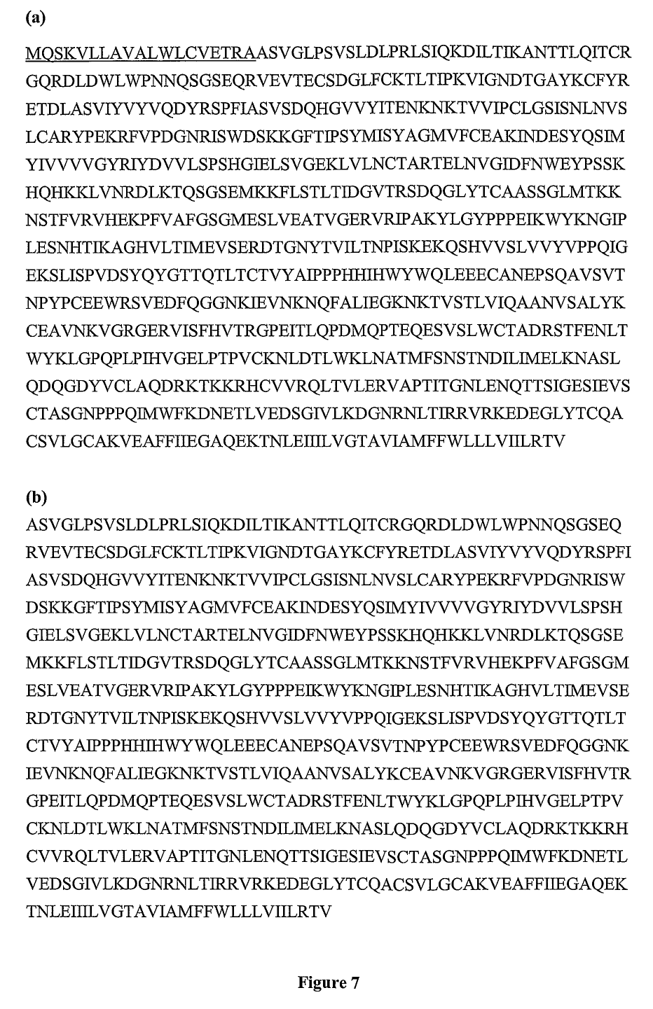

FIG. 7 shows the amino acid sequence of the KDR extracellular and transmembrane regions with, panel (a) (SEQ ID NO:26), and without a leader sequence, panel (b) (SEQ ID NO:27).

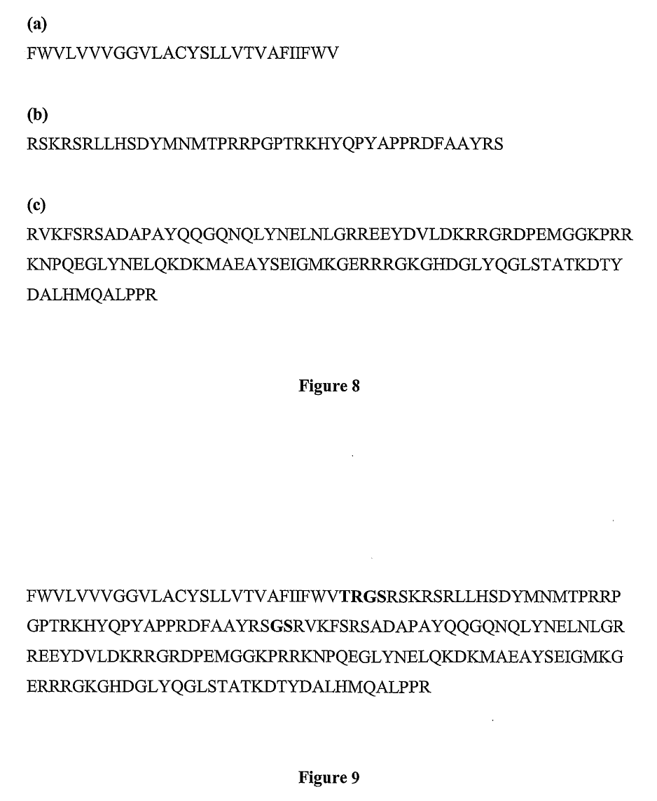

FIG. 8 shows the amino acid sequence of the used, panel (a) (SEQ ID NO:28), CD28 intracellular region, panel (b) (SEQ ID NO:29) and TCR ζ intracellular region panel (c) (SEQ ID NO:30).

FIG. 9 shows the amino acid sequence of CD28 transmembrane region, CD28 intracellular region and TCR ζ intracellular region linked by spacer sequences shown in bold typeface (SEQ ID NO:31).

### EXAMPLES

### Example 1

### Construction of Bioassay Receptor Expression Cloning Cassette and Shuttle Vector

To facilitate the construction of different bioassay receptors, an intermediate shuttle vector was designed. This shuttle vector contains the entire expression cassette necessary for the expression of the bioassay receptor. This vector includes the cloning cassette devised in pBluescript SK(+) (Stratagene) described previously in J. Immunol. 2004, 172:104. 5′ to this cloning cassette is the HCMV promoter, and the SV40 polyadenylation signal is 3′ to this cloning cassette. The cloning cassette consists of an extracellular domain (ECD) binding component (extracellular ligand-binding region), a transmembrane component and a signalling region component, and facilitates easy exchange of each individual component.

Combining the following DNA fragments generated the shuttle vector:
(a) the vector backbone of pBluescript II SK(+) (Stratagene) on a NotI to XhoI fragment;
(b) the cloning cassette described above and in FIG. 1 on a HindIII to EcoRI fragment;
(c) the HCMV promoter on a NotI to HindIII fragment; and
(d) the SV40 polyadenylation signal on an EcoRI to XhoI fragment.

This shuttle vector was used to generate various different bioassay receptors from the binding, transmembrane and signalling component fragments described in Example 2. The entire bioassay receptor expression cassette was then subcloned into a reporter gene vector as described in Example 3.

### Example 2

### Construction of Binding, Transmembrane and Signalling Region Fragments

### A) Human IL-17 Receptor Extracellular Ligand-Binding Region as a HindIII to NarI Fragment

A fragment comprising the leader sequence and extracellular region residues 1 to 320 (GenBank ref: NM 014339) of the human IL-17 receptor were PCR cloned with oligos:5′-cgggaagcttccaccatgggggccgcacgcagcccgccgtccgctgtcccggggcccctgctgggg ctgctcctgctgctcctgggcgtgctggccccgggtggtgcctccctgcgactcctggaccac-3′ (SEQ ID NO:22) and 5′-cccggcgccccacaggggcatgtagtccggaattgg-3′ (SEQ ID NO:23) from a plasmid containing the full length human IL-17 receptor gene. The SEQ ID NO:22 oligo introduces a 5′ HindIII site and Kosak sequence, and removes a NarI restriction site. SEQ ID NO:23 oligo introduces a 3′ NarI site. The PCR product was then digested with restriction enzymes HindIII and NarI. The amino acid sequence of IL-17 receptor used is shown in FIG. 6. The skilled person will understand that a different leader sequence which is usually cleaved on expression may be used and will be able to incorporate DNA encoding such a leader sequence using commonly known techniques.

### B) Human KDR Extracellular Ligand-Binding Region and Transmembrane Region as a HindIII to MluI Fragment

A fragment comprising the leader sequence, extracellular domain and transmembrane domain residues 1 to 789 (GenBank ref:NM 00002253) of the human kinase insert domain receptor (KDR) was generated synthetically (GENEART) with any natural internal HindIII, MluI, BamHI, EcoRI, BglII, NarI, NotI and SpeI sites removed. The fragment was digested from the supplied plasmid (042016pPCR-Script) with restriction enzymes HindIII and MluI. The amino acid sequence of KDR used is shown in FIG. 7. The skilled person will understand that a different leader sequence which is usually cleaved on expression may be used and will be able to incorporate DNA encoding such a leader sequence using commonly known techniques.

### C) Human CD28 Transmembrane Region and Intracellular Signalling Region and Human TCRζ Intracellular Signalling Region as a NarI to EcoRI Fragment

A fragment comprising residues 135 to 202 of human CD28 transmembrane and intracellular signalling region and residues 31 to 142 of human TCRζ intracellular signalling region was digested from a plasmid previously described (J. Immunol. 2004, 172:104) with restriction enzymes NarI and EcoRI. The amino acid sequences of CD28 transmembrane region, CD28 intracellular signalling region and TCRζ intracellular signalling region used are shown in FIG. 8. The amino acid sequence of all three regions linked using including spacer sequences is shown in FIG. 9.

### D) Human CD28 Signalling Region and Human TCRζ Intracellular Signalling Region as a MluI to EcoRI Fragment

A fragment comprising residues 162 to 202 of human CD28 intracellular signalling region and residues 31 to 142 of human TCRζ intracellular signalling region was digested from plasmid previously described (J. Immunol. 2004 172: 104) with restriction enzymes NarI and EcoRI. The amino acid sequences of CD28 intracellular signalling region and TCRζ intracellular signalling region used are shown in FIG. 8.

### Example 3

### Construction of Bioassay Receptor Reporter Gene Vectors

The full length expression cassette for each bioassay receptor generated by combining the components described in Example 2 within the shuttle vector described in Example 1, were then subcloned into reporter gene vectors pNifty2-Luc or pNifty2-SEAP (Invivogen). The latter vectors contain either a luciferase reporter gene (pNifty2-Luc) or a secreted alkaline phosphatase reporter gene (pNifty2-SEAP) under control of a NF-κB inducible promoter. In addition to this they also contain the selectable marker Zeocin™ for selection in both E. coli and mammalian cells. The bioassay receptor expression cassette was removed from the shuttle vector (Example 1) on a NotI to NotI fragment and cloned into the NotI site of pNifty2-Luc or pNifty2-SEAP. The expression cassette can be cloned in either direction and examples of both were selected and analysed.

### Example 4

### Generation of Stable Bioassay Receptor Reporter Gene Cell Lines

Plasmid DNA of vectors generated as described in Example 3 were transfected into the human T cell leukaemia cell line, Jurkat E6.1 using the Amaxa Nucleofector device according to the manufacturers instructions (Amaxa Biosystems). Stable cell lines were then generated by culture in Zeocin™ at concentrations of 200, 300 or 400 μg/ml.

### Example 5

### Analysis of Anti-Human IL-17 Antibody Using a IL-17R/CD28-TCRζ Bioassay Receptor

A stable cell line expressing a bioassay receptor that comprises the human IL-17 receptor extracellular ligand-binding region component, human CD28 transmembrane and intracellular signalling region, and human TCRζ intracellular signalling region components was generated. To these cells, a titration of human IL-17 was added and the amount of luciferase produced determined 4 hours later with a LucLite assay kit (Perkin Elmer) according to the supplier's instructions (see FIG. 2). Analysis of various cell lines expressing the IL-17R/CD28-TCRζ bioassay receptor demonstrated that vectors comprising the bioassay receptor expression cassette in the opposite orientation to the reporter gene cassette were more efficient. A concentration of IL-17 was selected from this titration (FIG. 2) and used to assess the ability of an anti-IL-17 antibody to block Luciferase production via the IL-17R/CD28-TCRζ Bioassay receptor (see FIG. 3).

### Example 6

### Analysis of Anti-Human KDR Antibody Using a KDR/CD28-TCRζ Bioassay Receptor

A stable cell line expressing a bioassay receptor that comprises the human KDR (VEGF receptor) extracellular ligand-binding region component and transmembrane region, human CD28 intracellular signalling region, and human TCRζ intracellular signalling region components was generated. To these cells, a titration of human VEGF-A (hVEGF) was added and the amount of luciferase produced determined 4 hours later with a LucLite assay kit (Perkin Elmer) according to the supplier's instructions (see FIG. 4). Analysis of various cell lines expressing the KDR/CD28-TCRζ bioassay receptor demonstrated that vectors comprising the bioassay receptor expression cassette in the opposite orientation to the reporter gene cassette were more efficient. A concentration of hVEGF was selected from this titration (FIG. 4) and used to assess the ability of an anti-KDR antibody to block luciferase production via the KDR/CD28-TCRζ bioassay receptor (see FIG. 5).

1. A vector comprising a DNA sequence encoding a bioassay receptor, said bioassay receptor comprising:
(a) an extracellular ligand-binding region capable of binding to a ligand;
(b) a transmembrane region;
(c) one or more intracellular signalling regions capable of transmitting a signal wherein said extracellular ligand-binding region and intracellular signaling region are not naturally fused together; and
(d) a reporter region;
wherein when said DNA sequence is expressed in a selected host cell under conditions suitable for vector expression, the binding of a ligand to the extracellular ligand-binding region results in the generation of a detectable signal from the reporter region.

2. The vector of claim 1, wherein the extracellular ligand-binding region is derived from a cytokine receptor, a cell surface receptor, or a soluble protein.

3. The vector of claim 2, wherein the cytokine receptor is IL-17R.

4. The vector of claim 2, wherein the cell surface receptor is KDR.

5. The vector of claim 1, wherein the DNA sequence encodes two intracellular signalling regions, one of said regions being derived from CD28.

6. The vector according to claim 5, wherein the second intracellular signalling region is derived from TCRζ.

7. The vector of claim 5, wherein the second intracellular signalling region is derived from a synthetic signaling region.

8. The vector of claim 7, wherein the synthetic signalling region is based on an ITAM.

9. The vector of claim 1, wherein the transmembrane region is derived from CD28.

10. The vector of claim 1, wherein the reporter region is luciferase.

11. The vector of claim 1, wherein the reporter region is SEAP.

12. A mammalian host cell comprising a vector as defined in claim 1.

13. A mammalian host cell comprising a vector as defined in claim 1, said host cell additionally comprising a second vector comprising a DNA sequence encoding a second bioassay receptor, said second bioassay receptor comprising:
(a) an extracellular ligand-binding region;
(b) a transmembrane region;
(c) one or more intracellular signaling regions capable of transmitting a signal wherein said extracellular ligand-binding region and intracellular signalling region are not naturally fused together; and optionally
(d) a reporter region;
wherein when said DNA sequence encoding said second bioassay receptor is expressed in a selected host cell under conditions suitable for vector expression, the binding of a ligand to the extracellular ligand-binding region results in the generation of a signal from the intracellular signalling region and, where present, a detectable signal is generated by the reporter region.

14. The mammalian cell according to claim 13, wherein said cell is a human Jurkat cell.

15. A polypeptide comprising:
(a) an extracellular ligand-binding region capable of binding to a ligand;
(b) a transmembrane region;
(c) one or more intracellular signalling regions capable of transmitting a signal wherein said extracellular ligand-binding region and intracellular signalling region are not naturally fused together; and
(d) a reporter region;
wherein the binding of a ligand to the extracellular ligand-binding region results in the generation of a detectable signal from the reporter region.

16. An in vitro method for assessing a compound of interest comprising:
(a) providing a mammalian host cell according to claim 13;
(b) providing a first sample comprising a ligand;
(c) providing a second sample comprising a compound of interest; and
(d) measuring a signal generated by the intracellular signalling region or regions and/or a detectable signal generated by the reporter region or regions.

17. The method of claim 16, additionally comprising the step of measuring a signal generated by the intracellular signalling region or regions and/or a detectable signal generated by the reporter region or regions before the provision of the second sample of part (c).

18. The method of claim 16, wherein the compound of interest is an antibody.
